(19) United States
(12) Patent Application Publication
(10) Pub. No.: US 2026/0199033 A1
(43) Pub. Date: Jul. 16, 2026

(54) ROBOT SYSTEM FOR GASTROSCOPY BASED ON VISUAL-TACTILE FUSION AND OPTICAL LEARNING

(71) Applicant: Zhejiang University, Hangzhou (CN)

(72) Inventors: Hong YU, Hangzhou (CN); Xin YU, Hangzhou (CN); Hongyuan LV, Hangzhou (CN); Zhichao AI, Hangzhou (CN); Hongxia XU, Hangzhou (CN); Xiaolong MA, Hangzhou (CN); Xiuying SUN, Hangzhou (CN)

(73) Assignee: Zhejiang University, Hangzhou (CN)

(21) Appl. No.: 19/190,646

(22) Filed: Apr. 27, 2025

(30) Foreign Application Priority Data
Jan. 16, 2025 (CN) 202510073131.0

(51) Int. Cl. A61B 34/30; A61B 1/00; A61B 1/07; A61B 1/273; A61B 34/00

(52) U.S. Cl. CPC A61B 34/30; A61B 1/00149; A61B 1/0016; A61B 1/07; A61B 1/2736; A61B 34/76; A61B 2034/301

(57) ABSTRACT

A robot system for gastroscopy based on visual-tactile fusion and optical learning includes a gastroscope control system, an identification and positioning module, and an integration computation module, wherein the gastroscope control system is configured to acquire gastroscope image data; the identification and positioning module is configured to receive the gastroscope image data, and perform region of interest (ROT) localization on the gastroscope image data; the integration computation module is configured to conduct molecular spectral measurement on the ROT to obtain the spectral data of gastric tissue, and calculate an integral energy ratio based on the spectral data. The present disclosure enables high-efficiency control of endoscope movement, alleviate the surgeons' workload, and incorporates advanced sensors and actuators, exhibiting high reliability and stability.

### CROSS REFERENCE TO THE RELATED APPLICATIONS

This application is based upon and claims priority to Chinese Patent Application No. 202510073131.0, filed on January 16, 2025, the entire contents of which are incorporated herein by reference.

### TECHNICAL FIELD

The present disclosure relates to the technical field of medical devices, and in particular to a robot system for gastroscopy based on visual-tactile fusion and optical learning.

### BACKGROUND

Upper gastrointestinal diseases are a major global health challenge. In particular, gastric cancer ranks fifth in morbidity and third in mortality rates among all cancers worldwide, respectively. Gastroscopy is regarded the gold standard for diagnosing and screening of various gastric disorders.

During gastroscopy, a surgeon typically holds the insertion part of the gastroscope in one hand while manipulating the operation part with the other, maintaining the position and angle of the gastroscope within the digestive tract for an extended period. Prolonged holding or maintaining an uncomfortable posture can easily lead to the surgeon's fatigue, thus affecting the accuracy of the examination and increasing the risk of missed diagnosis or misdiagnosis. In routine clinical practice, surgeons mainly rely on visual feedback alone, particularly in endoscopic treatment, where a lack of experience may lead to misjudgment, while positioning errors caused by hand tremors or unintended movements may increase the likelihood of iatrogenic injuries to patients. Therefore, replacing manual operation of the endoscope and integrating AI-based high precision positioning and recognition technology are of great significance. Existing patents have proposed a kind of gastroscopy holders, which partially free the surgeon's hands from manual operation. However, these holders on the market still have limitations. For instance, surgeons still need to directly hold the operating part of the gastroscope to adjust its position and angle, making the procedure cumbersome and inefficient.

Therefore, how to replace the surgeons' manual operations of the endoscope while enabling accurate image positioning and recognition remains a significant challenge to be addressed by those skilled in the art.

### SUMMARY

In view of the foregoing, the present disclosure provides a robot system for gastroscopy based on visual-tactile fusion and optical learning, to address the issues existing in the aforementioned background.

To achieve the above effects, the present disclosure adopts the following technical solutions.

A robot system for gastroscopy based on visual-tactile fusion and optical learning includes a gastroscope control system, an identification and positioning module, and an integration computation module, wherein the gastroscope control system acquires gastroscope image data; the identification and positioning module receives the gastroscope image data, and performs region of interest (ROI) localization on the gastroscope image data; and the integration computation module conducts molecular spectral measurement on the ROI to obtain the spectral data of gastric tissue, and calculates an integral energy ratio based on the spectral data.

Preferably, the gastroscope control system includes a gastroscope, a robotic arm, a miniature force sensor, a knob actuator and a main unit, wherein the gastroscope is arranged at a distal end of the robotic arm and is linearly connected to the main unit, to acquire the gastroscope image data; the robotic arm is linearly connected to the main unit, and a motion of the robotic arm is controlled by a pre-programmed algorithm within the main unit; the miniature force sensor is arranged at a distal end of the gastroscope, and when the distal end of the gastroscope contacts and presses against the gastrointestinal wall, the miniature force sensor generates force feedback signals and transmits the force feedback signals to the main unit; and the knob actuator controls an angle of the gastroscope image.

Preferably, the gastroscope includes an operation section, an insertion section, an optical fiber lens, an objective lens, and an image sensor, wherein the insertion section is a tubular structure equipped with optical fibers, the operation section is fixedly connected to the insertion section, an instrument inlet is disposed at a junction between the operation section and the insertion section; an opposite end of the operation section is arranged with a large knob and a small knob, the large knob and the small knob are coaxially arranged and connected to a guidewire within the insertion section; a curvature section is arranged near a distal end of the insertion section, the guidewire is arranged at an interior of the insertion section, and the curvature section at the distal end of the insertion section is driven by twisting the guidewire via the large knob and the small knob; the optical fiber lens, an instrument outlet, the objective lens and the image sensor are arranged at the distal end of the insertion section, wherein the optical fiber lens transmits light emitted from a light source, the objective lens captures the optical signal reflected from the gastrointestinal wall and performs imaging, and gastroscope image data is transmitted to a main unit through the image sensor; an endoscopic treatment instrument inserted from the instrument inlet and extends from the instrument outlet.

Preferably, the robotic arm includes position sensors, force sensors, motors, a base, two connecting rods, three joints, a gripper and a main unit connection interface, wherein the base is integrated with joints, and the connecting rods are arranged alternately with the joints, the gripper is arranged at a distal end of the joint farthest from the base, and a motor, a position sensor and a force sensor are arranged at each joint, the motor is configured to control rotational motion of the joint, the position sensor is configured to detect the motor's rotation angle in real time, and the force sensor is configured to measure the motor's torque output in real time; and the main unit connection interface is connected to the main unit by a cable.

Preferably, the main unit includes a light source, a display device, a control console, an image processor, a force feedback processor, a visual-tactile fusion system, and a motion control system of the gastroscope, wherein the light source transmits an optical signal to the optical fiber lens; the display device displays image signals output by the image processor; the image processor receives and processes the gastroscope image data collected by the image sensor; the control console generates control signals for the motion control system of the gastroscope; the force feedback processor processes force feedback signals collected by the miniature force sensor, and constructs a 3D safety spatial model based on a pre-programmed haptic database; the visual-tactile fusion system integrates the gastroscope image data with the force feedback signal, monitors real- time position and orientation tracking of the endoscope within the gastric cavity, and calculates pressure exerted by the objective lens on the gastrointestinal wall; and the motion control system receives signals from the control console, converts the signals into a motion command using a pre- programmed control algorithm, and transmits the command to the knob actuator and the robotic arm.

Preferably, the knob actuator is internally formed with mating recesses corresponding to the large knob and small knob and includes an outer turntable, an inner turntable, a first servo motor, a second servo motor, a central shaft and a housing, wherein the housing, the outer turntable and the inner turntable are coaxially arranged, the central shaft is fixed within the housing; the first servo motor is housed within the outer turntable, the second servo motor is housed within the inner turntable; both the first servo motor and the second servo motor are engaged with the central shaft gear to enable coaxial rotation; and the outer turntable drives the large knob to rotate, and the inner turntable drives the small knob to rotate.

Preferably, the knob actuator is cylindrical.

As demonstrated by the aforementioned technical solutions, compared with the prior art, the present disclosure provides a robot system for gastroscopy based on visual-tactile fusion and optical learning. The system enables high-efficiency control of endoscope movement, and alleviates the surgeons' workload; the system automatically executes a plurality of complex tasks, thereby optimizing the time and effort expenditure of surgeons; the system employs advanced sensors and actuators, exhibiting high reliability and stability; and the system serves as a foundation for subsequent remote endoscopic procedures, enabling motion control in response to remote network commands.

### BRIEF DESCRIPTION OF THE DRAWINGS

In order to more clearly illustrate the embodiments of the present disclosure or technical solutions in the related art, the accompanying drawings used in the embodiments or the related art will now be described briefly. It is obvious that the drawings in the following description are only the embodiment of the disclosure, and that those skilled in the art can obtain other drawings from these drawings without any creative efforts.

FIG. 1 is a structure diagram provided by the present disclosure; and

FIG. 2 is a schematic diagram of a knob actuator provided by the present disclosure.

List of reference characters: 1 gastroscope; 2 robotic arm; 3 knob actuator; 4 miniature force sensor; 5 main unit; 11 operation section; 12 insertion section; 13 large knob; 14 small knob; 15 curvature section; 16 instrument inlet; 17 instrument outlet; 18 optical fiber lens; 19 objective lens; 191 image sensor; 21 base; 22 joint; 221-motor; 222-position sensor; 223-force sensor; 23 connecting rod; 24 gripper; 31 outer turnplate; 32 inner turnplate; 331 first servo motor; 332 second servo motor; 34 central shaft; 35 housing; 51 light source; 52 display device; 53 control console; 54 image processor; and 55 force feedback processor.

### DETAILED DESCRIPTION OF THE EMBODIMENTS

Hereinafter, the technical solutions in the embodiments of the present disclosure will be described with reference to accompanying drawings. It is apparent that the described embodiments are only a part of the embodiments of the present disclosure, rather than all of the embodiments. Based on the embodiments of the present invention, all other embodiments that can be derived by those skilled in the art without any creative efforts shall fall within the scope of the present disclosure.

The present embodiment discloses a robot system for gastroscopy based on visual-tactile fusion and optical learning, including: a gastroscope control system, an identification and positioning module, and an integration computation module, wherein the gastroscope control system is configured to acquire gastroscope image data; the identification and positioning module is configured to receive the gastroscope image data, and perform region of interest (ROI) localization on the gastroscope image data; and the integration computation module is configured to conduct molecular spectral measurement on the ROI to generate the spectral data of gastric tissue, and calculate an integral energy ratio based on the spectral data.

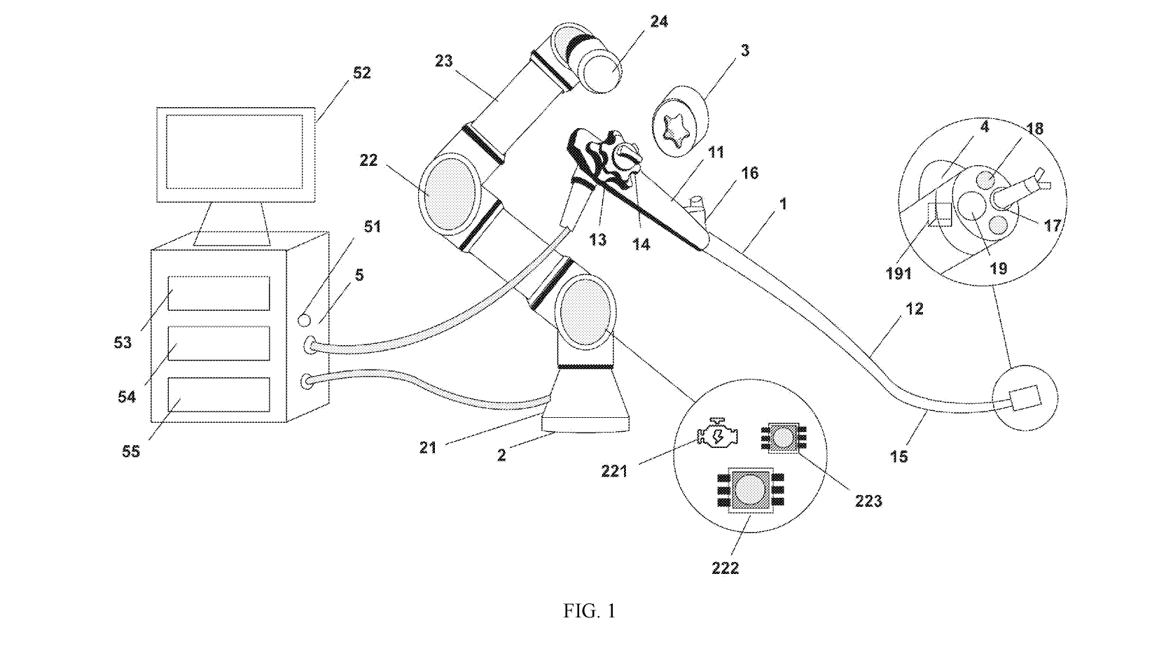

In a specific embodiment, as shown in FIG. 1, the gastroscope control system includes a gastroscope 1, a robotic arm 2, a knob actuator 3, a miniature force sensor 4, and a main unit 5, wherein the gastroscope 1 is arranged at a distal end of the robotic arm 2 and is linearly connected to the main unit 5, to acquire the gastroscope image data; the robotic arm 2 is linearly connected to the main unit 5, and the motion of the robotic arm 2 is controlled by a pre-programmed algorithm within the main unit 5; the knob actuator 3 is arranged on a large knob and a small knob (detachable), to control an angle of the gastroscope image; the miniature force sensor 4 is arranged at a distal end of the gastroscope 1, and when the distal end of the gastroscope 1 contacts and presses against the gastrointestinal wall, the miniature force sensor generates a force signal and transmits the force signal to the main unit 5.

In a specific embodiment, the gastroscope 1 includes an operation section 11, an insertion section 12, an optical fiber lens 18, an objective lens 19, and an image sensor 191, wherein the insertion section 12 is a tubular structure equipped with optical fiber lens, the operation section 11 is fixedly connected to the insertion section 12, an instrument inlet 16 is arranged at a junction between the operation section 11 and the insertion section 12, which is used to penetrate into the patient's alimentary canal, and endoscopic treatment instruments (e.g., grasping forceps and the like) enter through the inlet; an opposite end of the operation section is arranged with a large knob 13 and a small knob 14, the large knob 13 and the small knob 14 are coaxially arranged, and connected to a guidewire within the insertion section 12; a curvature section 15 is arranged near a distal end of the insertion section 12, the guidewire is arranged at an interior of the insertion section 12, and connected to the large knob 13 and the small knob 14, the curvature section 15 at the distal end of the insertion section 12 is driven by twisting the guidewire via the large knob 13 and the small knob 14, to adjust a rotation direction of the gastroscope lens; the optical fiber lens 18, an instrument outlet 17, the objective lens 19 and the image sensor 191 are arranged at the distal end of the insertion section 12, the optical fiber lens 18 transmits light emitted from a light source 51, the optical fiber lens 18 is at a distal end of the optical fibers, transmitting the light emitted from the light source to illuminate the interior of the gastrointestinal tract, the objective lens 19 accepts the optical signal reflected by the gastrointestinal wall and performs imaging, and the gastroscope image data is transmitted to the main unit 5 through the image sensor 191; and an endoscopic treatment instrument enters from the instrument inlet 16 and extends from the instrument outlet 17, (such as grasping forceps and the like), entering into the gastrointestinal tract through the outlet for performing operations such as biopsy.

In a specific embodiment, the robotic arm 2 includes position sensors, force sensors, motors, a base 21, two connecting rods 23, three joints 22, a gripper 24 and a main unit connection interface, the base 21 is fixed with joints 22, the connecting rods 23 and the joints 22 are arranged alternately, and the gripper 24 is arranged at a distal end of the joint 22 farthest from the base 21, the gripper 24 may be expressed as a hand-held style robotic arm, and may also be expressed as a splint style structure. No matter what kind of structural style, the gripper 24 fits with the operation section 11, which is configured to support and fix gastroscope 1, so as to facilitate the movement and attitude transformation of the robotic arm 2; a motor (so that the gastroscopy 1 may complete forward, backward, external rotation, internal rotation and other actions), a position sensor and a force sensor (being installed on the connecting shaft of the motor, to acquire a rotation angle the motor and a motor torque in real time, and transmit a position signal and a force signal formed to the main unit 5), the motor is configured to control a twisting motion of the joint 22, the position sensor is configured to acquire the rotation angle of the motor in real time, and the force sensor is configured to acquire the motor torque in real time; and the main unit connection interface connects to the main unit 5 through a cable, configured to transmit the position signal and the force signal, and to receive a motion control command.

In a specific embodiment, the main unit 5 includes a light source 51, a display device 52, a control console 53, an image processor 54, a force feedback processor 55, a visual-tactile fusion system, and a motion control system of the gastroscope, wherein the light source 51 is a cold light source, which is configured to output an optical signal through the optical fibers to the optical fiber lens 18; the display device 52 is configured to display image signals output by the image processor 54, and present the image signals in the form of images, videos, or the like; the image processor 54 is configured to receive and process the gastroscope image data collected by the image sensor 191, and is capable of decomposing the image data into signals including a position signal, a region signal, a lesion signal, a posture signal, and the like, thereby constructing an identification and positioning model and generating visualized image signals for output to the display device 52; at the same time, the image processor 54 includes a software part, namely a high-precision tissue identification and localization algorithm based on artificial intelligence (as detailed below), which may generate signals such as the position signal, the region signal, the lesion signal, the posture signal and the like, form the identification and positioning model, and generate a three-dimensional image displayed on the display device 52; the control console 53 is a surgeons-operated control platform, configured to input motion instructions including forward, backward, and the like, so as to facilitate remote, contact-free, and accurate gastroscopic manipulation, and to send signals to the motion control system of the gastroscope; the force feedback processor 55 is configured to receive force feedback signals collected by the miniature force sensor 4 and construct a safety and spatial mode based on a predefined tactile database (including safety thresholds and spatial tactile characteristics of different locations); the visual-tactile fusion system, being a software component, is configured to integrate characteristics of distinct properties from visual-tactile signals in a phased manner, and calculate a correlation between visual features and tactile features, achieving a high-quality fusion of visual and tactile features with a maximum correlation coefficient as an index, so that the position and orientation of the endoscope in the stomach may be tracked in real time, the pressure of the objective lens on the gastrointestinal wall is determined, which may be displayed in the display device 52, and provide early warning and navigation. the motion control system of the gastroscope receives the signal of the control console 53, and converts the signal into a motion command through a preset control algorithm and sends the motion command to the knob actuator 3 and the robotic arm 2. The motion control system the gastroscope is a software section, mainly used for controlling algorithms, including a 2-layer closed-loop control structure: an inner layer is a closed-loop control of the desired attitude and the actual attitude feedback of the lens, and an outer layer is the control of the output torque of the motor and the feedback torque of the connecting shaft.

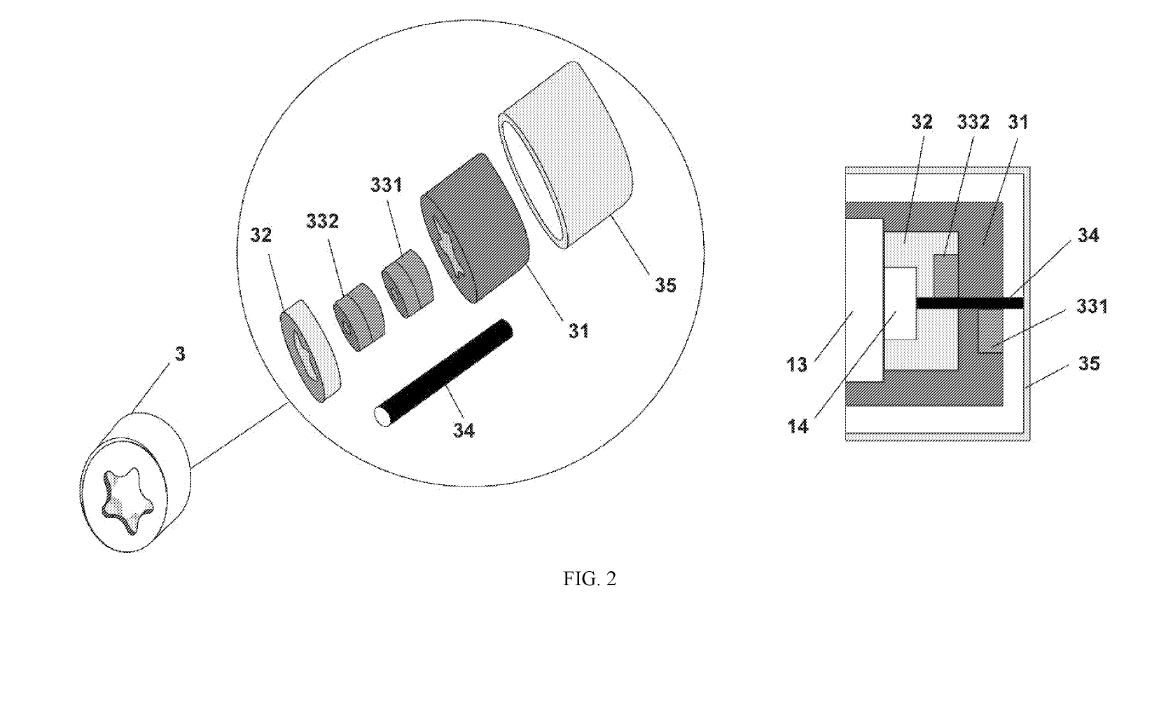

In a specific embodiment, as shown in FIG. 2, the knob actuator 3 is internally formed with mating recesses corresponding to the large knob 13 and small knob 14 and includes an outer turntable 31, an inner turntable 32, a first servo motor 331, a second servo motor 332, a central shaft 34 and a housing 35, wherein the housing 35, the outer turntable 31 and the inner turntable 32 are coaxially arranged, and the central shaft 34 is fixed within the housing 35; the first servo motor 331 is housed within the outer turntable 31, and the second servo motor 332 is housed within the inner turntable 32; both the first servo motor 331 and the second servo motor 332 are engaged with the central shaft 34 gear to enable coaxial rotation; and the outer turntable 31 drives the large knob 13 to rotate, and the inner turntable 32 drives the small knob 14 to rotate. The rotation of the outer turntable 31 and the inner turntable 32 does not interfere with each other. The first servo motor 331 and the second servo motor 332 may accept a motion signal provided by the main unit 5 and generate power, driving the outer turntable 31 to rotate in either a counterclockwise or clockwise direction, and driving the inner turntable 32 to rotate in either a counterclockwise or clockwise direction

In a specific embodiment, the knob actuator 3 is cylindrical.

The miniature force sensor 4 is a miniature device that is additionally mounted to the gastroscope 1, through various forms such as sticking, magnetic suction or clamping, the miniature force sensor may be placed at the end of the insertion part of gastroscope 12. When the gastroscope lens contacts and squeezes the inner wall of the gastrointestinal tract, a force signal is dynamically formed, and transmitted back to the main unit 5 through the internal Bluetooth wireless.

The high-precision tissue identification and localization algorithm based on artificial intelligence may use the continuous dynamic image data obtained by gastroscopy for data processing and feature extraction. Combined with the existing image identification and location algorithm for endoscope, training and refinement are performed, so that an algorithm after processing may accurately identify and locate the lesion features in a mixed background. The output of the algorithm may provide accurate target position information to the main unit 5, which lays an accurate positioning foundation for the robot system for the gastroscopy based on visuo- tactile fusion and optical learning. It mainly includes three parts.

### In the identification and positioning module: establishing endoscopic image and video data database

1. Data acquisition and pre-processing: Endoscopic equipment is used to obtain high- quality image and video data, and the standardization and quality control are ensured during data acquisition, such as calibration equipment and light source. Each data sample has detailed annotations, including location, size, severity and other information of the lesion, and through cooperation with clinicians, the accuracy of the annotations and clinical relevance is ensured. Because the data may have problems such as low contrast, illumination change, motion blur and noise, the data needs to be preprocessed to simplify the process of subsequent analysis and improve the robustness of the algorithm.

2. Feature extraction: there are many important feature information in the preprocessed endoscopic image and video data. These features may include the tissue structure, morphological characteristics, dynamic changes and movement characteristics of the lesion, and they are the basis of subsequent recognition and localization algorithms, which may help identify the region of interest and accurately locate it.

3. Database design and management: after data processing, it is necessary to establish a suitable database to save data and manage data, ensuring effective support for medical data storage, management and analysis needs, meanwhile guaranteeing data security and privacy protection, and complying with relevant regulations and standards.

### Establishing endoscopic image and video data database

1. Algorithm selection and optimization: the existing recognition and location models are developing rapidly, on the basis of previous studies, aspects of the model, including accuracy, recall ratio, processing speed and so forth are evaluated. Selecting models suitable for different scenarios for further analysis.

2. Algorithm optimization and expansion: in view of the unique characteristics and challenges of the gastric environment, such as lesion morphology, illumination changes and image noise from different perspectives, the algorithms in different scenarios are fine-tuned and extended, including optimizing the architecture of the model, adjusting the network depth, parameter settings and so forth, to improve the accuracy and stability of identification and positioning.

### Migration from algorithms to control system integration

1.Real-time processing system: endoscopy requires real-time feedback of positioning and recognition information, and requires comprehensive consideration of hardware acceleration, efficient algorithms, stream processing architecture, and user interface design, and a high- performance real-time processing system suitable for endoscopy can be developed to ensure that medical personnel can quickly obtain and analyze key identification and positioning information.

2. System integration: the verified organ identification and localization model and real- time processing system are integrated into the gastroscopy robot system, ensuring that the gastroscope robot has accurate positioning recognition technology and real-time processing ability.

In the integral calculation module: based on the precise diagnosis and treatment technology of gastric lesions of optical-Raman spectroscopy, the method of combining fiber Raman spectroscopy system and integral energy ratio is used to quickly locate gastric lesions, molecular spectroscopy of gastric tissue was measured, the spectral data of gastric tissue were obtained, and real-time analysis and non-invasive diagnosis are performed. The Raman spectrometer, Raman light source, Raman fiber probe and Raman data processing equipment are included. Effectively integrating different components, ensuring that images of internal tissue results, contact mechanics data, and atlas data may be collected simultaneously during gastroscopy, and ensuring the stability and accuracy of the operation, so as to achieve real-time collection, analysis and diagnosis. It mainly includes two aspects.

### I. Establishing a Raman spectrum data set of gastric lesions

1. Collecting data: More than 30,000 spectral data of normal gastric tissue and abnormal gastric tissue are collected. The characteristic Raman spectra of normal gastric tissue and abnormal gastric tissue are obtained, aiming at the interference of Raman spectral fluorescence background on the detection results, a variety of data analysis methods are used to analyze and process the obtained tissue spectral data, including denoising, feature extraction, statistical analysis, classification, prediction, etc.

2. Establish a Raman spectrum data set of gastric lesions: the original Raman spectrum is processed and converted by fast Fourier transform, the Raman spectrum features were extracted and the Raman spectrum data set of gastric lesions is established. According to the collected Raman spectra, the processed Raman spectra are compared with the Raman spectra data set, ratios of integral and energy of normal spectra and lesion spectra are obtained, respectively, and the ratios of integral to energy are used as indexes to be used as a reference for medical staff.

### II. Spectral measurement and data processing

Using the biopsy hole of the gastroscope, the optical fiber Raman technique is used for gastroscopy, at the same time, molecular spectroscopy of gastric tissue is measured, to obtain spectral data of normal and diseased gastric tissues. Using clustering analysis, principal component analysis, support vector machine, neural network algorithm, etc., a mathematical model for the identification of specific types of spectral features is established, to accurately diagnose gastric cancer lesions and biopsy surgical margins.

Various embodiments of the present specification are described in a progressive manner, and each embodiment focuses on the description that is different from the other embodiments, and the same or similar parts between the various embodiments are referred to with each other. For the device disclosed by the embodiments, because the device corresponds to the method disclosed by the embodiment, the description is relatively simple, and the relevant points may refer to a partial description of the method.

The above description of the disclosed embodiments enables those skilled in the art to implement or use the present disclosure. Various amendments to the embodiments will be apparent to those skilled in the art. The general principles defined herein may be implemented in other embodiments without departing from the spirit or scope of the disclosure. Therefore, the present disclosure will not be limited to the embodiments shown herein but is to be accorded the widest scope consistent with the principles and novel features disclosed herein.

What is claimed is:

1. A robot system for a gastroscopy based on a visual-tactile fusion and an optical learning, comprising: a gastroscope control system, an identification and positioning module, and an integration computation module, wherein the gastroscope control system acquires gastroscope image data; the identification and positioning module receives the gastroscope image data, and performs a region of interest (ROT) localization on the gastroscope image data; and the integration computation module conducts a molecular spectral measurement on an ROT to obtain spectral data of a gastric tissue, and calculates an integral energy ratio based on the spectral data.

2. The robot system according to claim 1, wherein the gastroscope control system comprises a gastroscope, a robotic arm, a miniature force sensor, a knob actuator, and a main unit, wherein the gastroscope is arranged at a distal end of the robotic arm and is linearly connected to the main unit, to acquire the gastroscope image data; the robotic arm is linearly connected to the main unit, and a motion of the robotic arm is controlled by a pre-programmed algorithm within the main unit;
the miniature force sensor is arranged at a distal end of the gastroscope, and when the distal end of the gastroscope contacts and presses against a gastrointestinal wall, the miniature force sensor generates a force feedback signal and transmits the force feedback signal to the main unit; and the knob actuator controls an angle of a gastroscope image.

3. The robot system according to claim 2, wherein the gastroscope comprises an operation section, an insertion section, an optical fiber lens, an objective lens, and an image sensor, wherein the insertion section is a tubular structure equipped with an optical fiber, the operation section is fixedly connected to the insertion section, and an instrument inlet is disposed at a junction between the operation section and the insertion section; an opposite end of the operation section is arranged with a first knob and a second knob, the first knob is larger than the second knob, and the first knob and the second knob are coaxially arranged and connected to a guidewire within the insertion section; a curvature section is arranged near a distal end of the insertion section, the guidewire is arranged at an interior of the insertion section, and the curvature section at the distal end of the insertion section is driven by twisting the guidewire via the first knob and the second knob; the optical fiber lens, an instrument outlet, the objective lens, and the image sensor are arranged at the distal end of the insertion section, wherein the optical fiber lens transmits a light emitted from a light source, the objective lens captures an optical signal reflected from the gastrointestinal wall
and performs an imaging, and the gastroscope image data is transmitted to the main unit through the image sensor; and an endoscopic treatment instrument is inserted from the instrument inlet and extends from the instrument outlet.

4. The robot system according to claim 3, wherein the robotic arm comprises a position sensor, a force sensor, a motor, a base, two connecting rods, three joints comprising a first joint, a second joint and a third joint, a gripper, and a main unit connection interface, wherein the base is integrated with the first joint, the two connecting rods are arranged alternately with the three joints, and the gripper is arranged at a distal end of the third joint farthest from the base; the motor, the position sensor, and the force sensor are arranged at each of the three joints, the motor is configured to control a rotational motion of each of the three joints, the position sensor is configured to detect the a rotation angle of the motor in real time, and the force sensor is configured to measure a torque output of the motor in real time; and the main unit connection interface is connected to the main unit by a cable.

5. The robot system according to claim 4, wherein the main unit comprises the light source, a display device, a control console, an image processor, a force feedback processor, a visual-tactile fusion system, and a motion control system of the gastroscope, wherein the light source transmits the optical signal to the optical fiber lens; the display device displays an image signal output by the image processor; the image processor receives and processes the gastroscope image data collected by the image sensor; the control console generates a control signal for the motion control system of the gastroscope; the force feedback processor processes the force feedback signal collected by the miniature force sensor, and constructs a 3D safety spatial model based on a pre- programmed haptic database; the visual-tactile fusion system integrates the gastroscope image data with the force feedback signal, monitors a real-time position and an orientation tracking of an endoscope within a gastric cavity, and calculates a pressure exerted by the objective lens on the gastrointestinal wall; and the motion control system receives the control signal from the control console, converts the control signal into a motion command using a pre-programmed control algorithm, and transmits the motion command to the knob actuator and the robotic ann.

6. The robot system according to claim 3, wherein the knob actuator is internally formed with mating recesses corresponding to the first knob and the second knob and comprises an outer turntable, an inner turntable, a first servo motor, a second servo motor, a central shaft, and a housing, wherein the housing, the outer turntable, and the inner turntable are coaxially arranged,
and the central shaft is fixed within the housing; the first servo motor is housed within the outer turntable, and the second servo motor is housed within the inner turntable; both the first servo motor and the second servo motor are engaged with a central shaft gear to enable a coaxial rotation;
and the outer turntable drives the first knob to rotate, and the inner turntable drives the second knob to rotate.

7. The robot system according to claim 6, wherein the knob actuator is cylindrical.
